(19) United States
(12) Patent Application Publication
(10) Pub. No.: US 2009/0131967 A1
(43) Pub. Date: May 21, 2009

(54) SURGICAL TROCAR

(76) Inventors: Jeffrey D. Hollis, Gallatin, TN (US); Gregory D. Hollis, Nashville, TN (US)

Correspondence Address: STURM & FIX LLP, 206 SIXTH AVENUE, SUITE 1213, DES MOINES, IA, 50309-4076, US

(21) Appl. No.: 11/941,258

(22) Filed: Nov. 16, 2007

(51) Int. Cl. A61B 17/34

(52) U.S. Cl. 606/185

(57) ABSTRACT

The present invention provides a surgical trocar including an elongated trocar passageway and an access channel disposed in spaced relationship to each other. The access channel includes a gas seal to prevent gas leakage while the access channel is in use, and a valve to close the access channel when not in use. Once, the trocar is inserted into the body cavity, the access channel allows access of catheters and or other small diameter devices without the need for making extra incisions. This allows continued use of the trocar while utilizing the access channel, giving it a dual function usage. The access channel allows devices to be angled away from the trocar once inside the body cavity making device manipulation easier.

### BACKGROUND OF THE INVENTION

1. Field of the Invention

The present invention relates to the field of surgical instruments, and more particularly to a surgical trocar having an access channel disposed in spaced relationship to the trocar passageway.

2. Description of Related Art

As can be seen by reference to the following U.S. Pat. Nos. 5,139,487; 5,743,881; 5,941,852; U.S. Publn. 20070016100; and EP0756505, the prior art is replete with myriad and diverse surgical trocars.

While all of the aforementioned prior art constructions are adequate for the basic purpose and function for which they have been specifically designed, they are uniformly deficient with respect to their failure to provide a simple, efficient, and practical trocar having an integrated access channel.

As a consequence of the foregoing situation, there has existed a longstanding need for a new and improved surgical trocar, and the provision of such a device is a stated objective of the present invention.

### BRIEF SUMMARY OF THE INVENTION

Briefly stated, the present invention provides a surgical trocar including an elongated trocar passageway and an access channel disposed in spaced relationship to each other. The access channel includes a gas seal to prevent gas leakage while the access channel is in use, and a valve to close the access channel when not in use. Once, the trocar is inserted into the body cavity, the access channel allows access of catheters and or other small diameter devices without the need for making extra incisions. This allows continued use of the trocar while utilizing the access channel, giving it a dual function usage. The access channel allows devices to be angled away from the trocar once inside the body cavity making device manipulation easier.

One embodiment of the trocar has the access channel integrally formed in a housing with the trocar passageway. Another embodiment of the trocar includes a sheath that receives the elongated housing of a conventional trocar where the sheath includes an access channel disposed in spaced relationship to the trocar passageway.

The objective of this invention is to provide a multipurpose access channel for a trocar, for use during laparoscopic surgery. Current laparoscopy trocars do not provide an access channel and thus require separate incisions if a small diameter device, such as a catheter, is to be used. This improves current trocars by making them more versatile and eliminates the need for excess incisions Currently, during laparoscopic cholecystectomy, a separate stab incision is required to insert a cholangiocatheter or stone retrieval device. This new access channel provides integrated access through the existing trocar, removing the need for extra incisions.

### BRIEF DESCRIPTION OF THE SEVERAL VIEWS OF THE DRAWINGS

These and other attributes of the invention will become more clear upon a thorough study of the following description of the best mode for carrying out the invention, particularly when reviewed in conjunction with the drawings, wherein:

FIG. 1 is a side elevational view of the surgical trocar of the present invention, with the proximal and distal ends cut away;

FIG. 2 is an enlarged partial sectional view of the proximal end of the trocar;

FIG. 3 is an enlarged partial sectional view of the distal end of the trocar;

FIG. 4 is a proximal end elevational view of the trocar;

FIG. 5 is a distal end elevational view of the trocar;

FIG. 6 is a side elevational view of a glove sheath for a conventional trocar, with the proximal and distal ends cut away;

FIG. 7 is an enlarged partial sectional view of the proximal end of the sheath;

FIG. 8 is an enlarged partial sectional view of the distal end of the sheath;

FIG. 9 is a proximal end elevational view of the sheath; and

FIG. 10 is a distal end elevational view of the sheath.

### DETAILED DESCRIPTION OF THE INVENTION

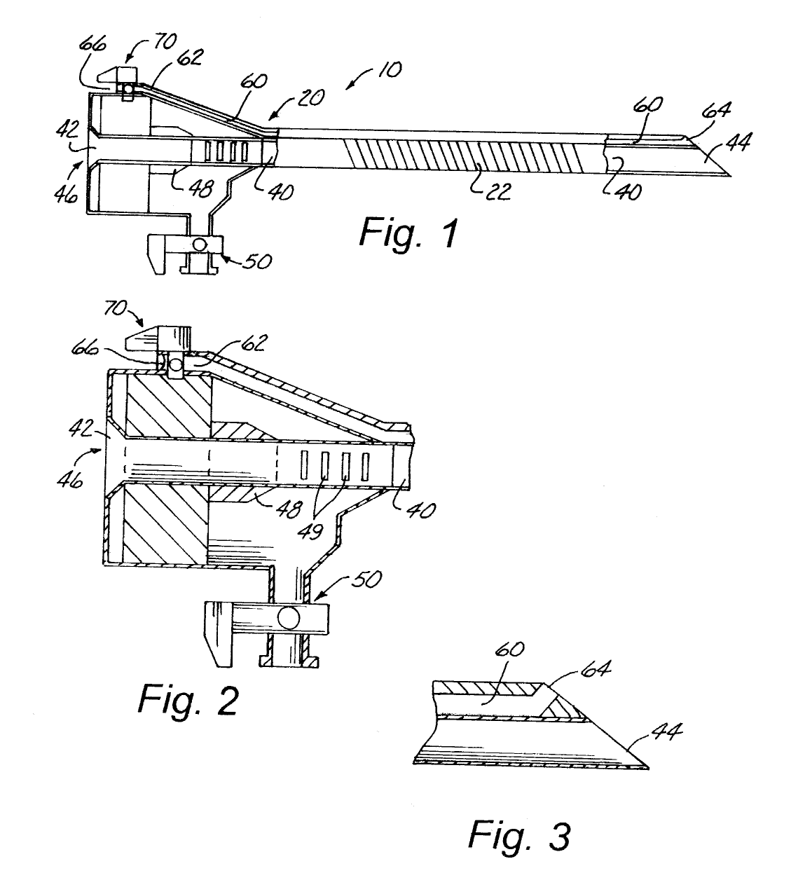

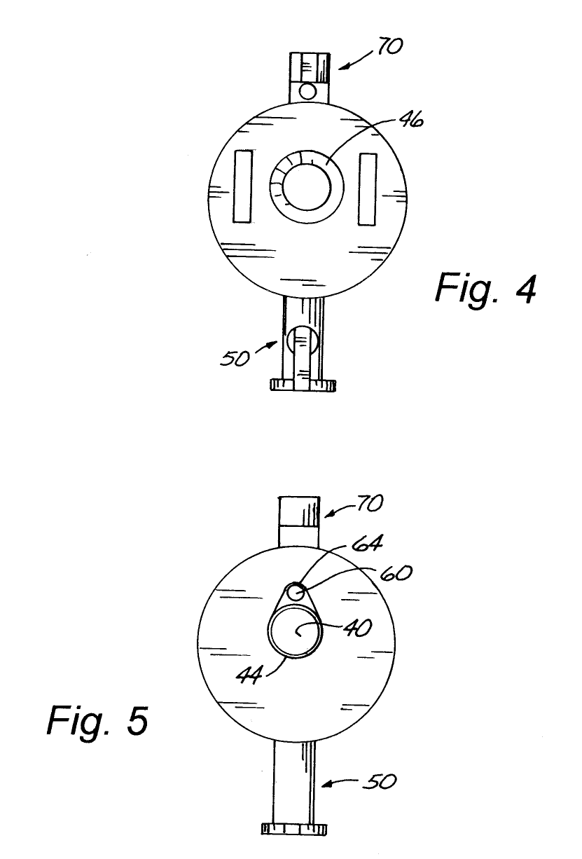

As can be seen by reference to the drawings, and in particular to FIG. 1, the surgical trocar that forms the basis of the present invention is designated generally by the reference number 10. The trocar 10 includes an elongated housing 20 with a trocar passageway 40 and an access channel 60 formed in the housing 20 in spaced relationship to each other. The exterior of the housing 20 has grooves 22 for tissue traction.

The trocar passageway 40 has a proximal end 42 and a distal end 44. The proximal end 42 is in open communication with the trocar collar 46 which includes a rubber boot gas seal 48 through which the instrument (not shown) is directed and internal gas inlets 49. A stopcock valve 50 is also in communication with the trocar passageway 40.

The access channel 60 has a proximal end 62 and a distal end 64. A diaphragm gas seal 66 is disposed near the proximal end 62 to prevent gas leakage out through the access channel 60 when it is in use. Also, a stopcock valve 70 is provided to close the access channel 60 when it is not in use. A portion of the access channel 60 at the distal end 64 is directed away from the longitudinal axis of the trocar passageway 40 so that devices carried through the access channel 40 may be easily manipulated without interference with the instrument introduced through the trocar passageway 40.

The invention may be used for different small diameter devices on different size trocars. The trocar function itself is not compromised. This access channel 60 can be used for diagnostic and therapeutic interventions depending on the device. Diagnostic uses, for example, would include a channel for cholangiography or liver biopsy. Therapeutic uses, for example, would include catheters for bile duct exploration such as stone baskets.

The trocar may be structured differently by changing the location of the access channel 60 either longitudinally or obliquely in reference to the trocar passageway 40. The access channel 60 can be structured to different diameters to accommodate different interventional devices. The opening of the channel 60 can be structured to open at the distal end 44 or anywhere along the trocar passageway 40. The trocar itself can be structured to different sizes depending on what access to a body cavity is needed. Multiple access channels can be placed on a single trocar. The channel can be incorporated into either cutting or dilating type trocars. The cross section of the housing 20 could be modified from the tear drop shape shown in FIG. 5 without changing the functionality.

The device works by not only allowing laparoscopic access to a body cavity, but also by providing a built in channel 60 for small diameter device access. The trocar passageway 40 allows laparoscopy instruments to be inserted into a body cavity for surgery. The integrated access channel 60 allows simultaneous use of the trocar 10 for other small diameter devices, such as cholangiocatheters, without compromising the use of the trocar passageway 40 for instruments. This design provides a versatile alternative to current trocars.

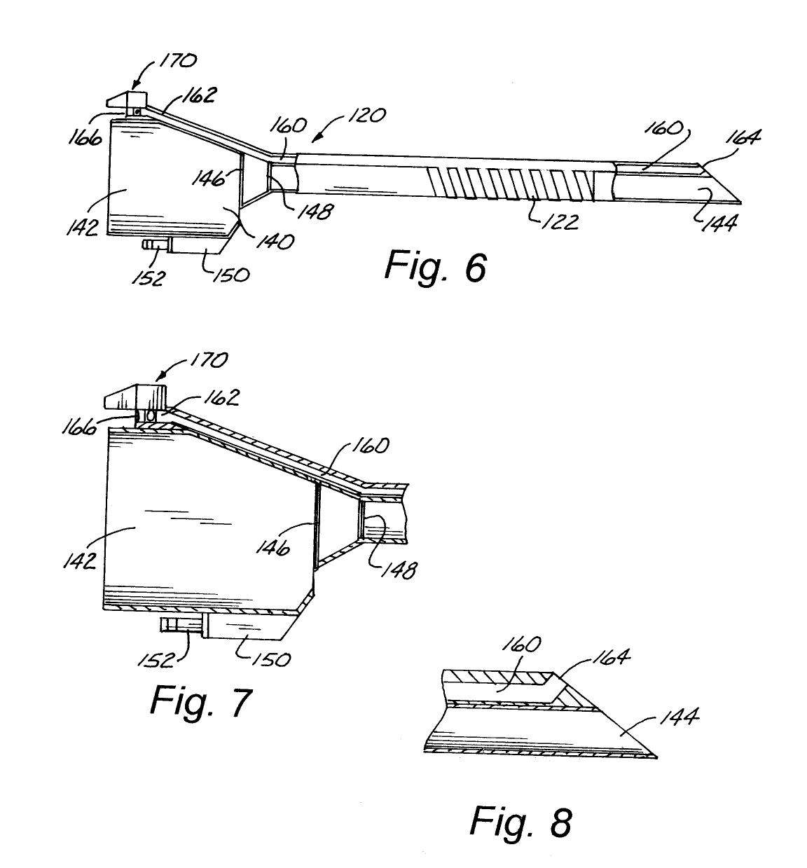

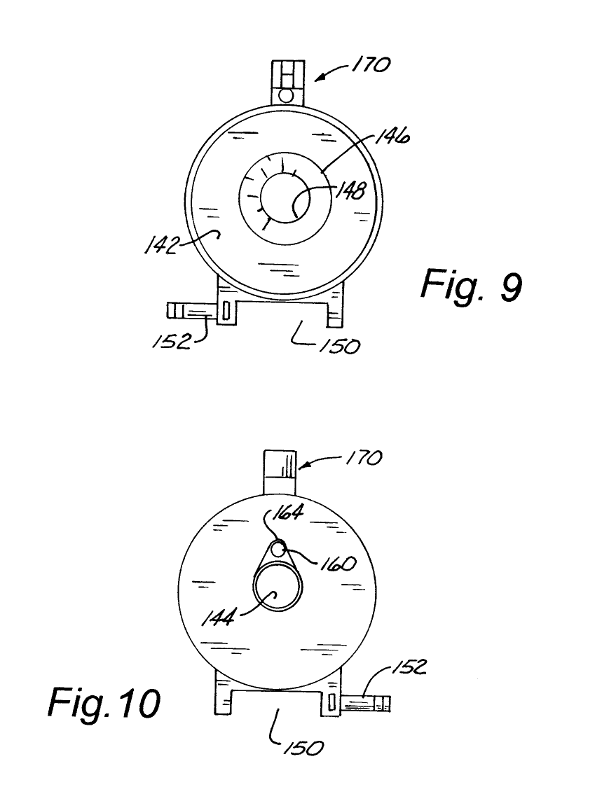

A second embodiment of the invention uses a glove sheath 120 as shown in FIGS. 6-10, in combination with a conventional trocar. The sheath 120 is disposed to matingly receive the housing of a conventional trocar. The sheath 120 includes a trocar receiving cavity 140 and an access channel 160 disposed in spaced relationship to the cavity 140. The exterior of the sheath 120 has grooves 122 for tissue traction.

The trocar cavity 140 has a proximal end 142 and a distal end 144. A pair of “O”-ring seals 146 and 148 prevents gas leakage and stabilizes the trocar within the cavity 140. A groove 150 receives the air insufflation port of the trocar, and a latch 152 secures the trocar within the sheath 120 at the air insufflation point.

The access channel 160 has a proximal end 162 and a distal end 164. A diaphragm gas seal 166 and a stopcock valve 170 are carried near the proximal end 162. The sheath 120 thus provides a retrofit of a conventional trocar that functions like the main embodiment of the present invention.

Although only an exemplary embodiment of the invention has been described in detail above, those skilled in the art will readily appreciate that many modifications are possible without materially departing from the novel teachings and advantages of this invention. Accordingly, all such modifications are intended to be included within the scope of this invention as defined in the following claims.

Having thereby described the subject matter of the present invention, it should be apparent that many substitutions, modifications, and variations of the invention are possible in light of the above teachings. It is therefore to be understood that the invention as taught and described herein is only to be limited to the extent of the breadth and scope of the appended claims.

I claim:

1. A surgical trocar comprising:
an elongated housing;
an elongated trocar passageway formed in the housing, the passageway having a proximal end, a distal end, and a longitudinal axis; and
an elongated access channel disposed in spaced relationship to the trocar passageway, the access channel having a proximal end and a distal end.

2. The surgical trocar of claim 1, further including a gas seal disposed within the access channel near its proximal end, whereby gas is prevented from exiting its proximal end while the access channel is in use.

3. The surgical trocar of claim 1, further including a valve disposed within the access channel near its proximal end, whereby the access channel may be closed when not in use.

4. The surgical trocar of claim 2, further including a valve disposed within the access channel near its proximal end, whereby the access channel may be closed when not in use.

5. The surgical trocar of claim 1, wherein the distal end of the access channel is inwardly spaced from the distal end of the trocar passageway.

6. The surgical trocar of claim 2, wherein the distal end of the access channel is inwardly spaced from the distal end of the trocar passageway.

7. The surgical trocar of claim 3, wherein the distal end of the access channel is inwardly spaced from the distal end of the trocar passageway.

8. The surgical trocar of claim 4, wherein the distal end of the access channel is inwardly spaced from the distal end of the trocar passageway.

9. The surgical trocar of claim 1, wherein a portion of the access channel adjacent its distal end is directed away from the longitudinal axis of the trocar passageway.

10. The surgical trocar of claim 2, wherein a portion of the access channel adjacent its distal end is directed away from the longitudinal axis of the trocar passageway.

11. The surgical trocar of claim 3, wherein a portion of the access channel adjacent its distal end is directed away from the longitudinal axis of the trocar passageway.

12. The surgical trocar of claim 4, wherein a portion of the access channel adjacent its distal end is directed away from the longitudinal axis of the trocar passageway.

13. The surgical trocar of claim 5, wherein a portion of the access channel adjacent its distal end is directed away from the longitudinal axis of the trocar passageway.

14. The surgical trocar of claim 6, wherein a portion of the access channel adjacent its distal end is directed away from the longitudinal axis of the trocar passageway.

15. The surgical trocar of claim 7, wherein a portion of the access channel adjacent its distal end is directed away from the longitudinal axis of the trocar passageway.

16. The surgical trocar of claim 8, wherein a portion of the access channel adjacent its distal end is directed away from the longitudinal axis of the trocar passageway.

17. The surgical trocar of claim 1, wherein the access channel is formed in the housing.

18. The surgical trocar of claim 1, wherein the access channel is formed in a sheath disposed to receive the elongated housing of the trocar.

19. The surgical trocar of claim 17, wherein the housing has a tear drop cross section.

20. The surgical trocar of claim 18, wherein the housing has a tear drop cross section.
